(19) United States
(12) Patent Application Publication
(10) Pub. No.: US 2017/0196801 A1
(43) Pub. Date: Jul. 13, 2017

(54) APPLICATION OF TOPICAL PRODUCT USING A SONIC DEVICE

(71) Applicant: Flexxsonic Corp., Mount Prospect, IL (US)

(72) Inventors: Kamyar Ghazvini, Mount Prospect, IL (US); Anna Ghazvini, Mount Prospect, IL (US)

(21) Appl. No.: 14/993,710

(22) Filed: Jan. 12, 2016

(51) Int. Cl. A61K 9/00; A61K 31/7004; A61K 31/10; A61K 36/886; A61K 36/16; A61K 36/258; A61K 36/48; A61K 36/899; A61K 36/28; A61K 36/77; A61K 36/185; A61K 31/198; A61K 31/122; A61K 36/53; A61K 36/537; A61K 31/355; A61K 31/519; A61K 31/216; A61K 31/665; A61K 9/06; A61H 23/02; A61K 31/165

(52) U.S. Cl. CPC A61K 9/0009; A61K 31/165; A61K 31/7004; A61K 31/10; A61K 36/886; A61K 36/16; A61K 36/258; A61K 36/48; A61K 36/899; A61K 36/28; A61K 36/77; A61K 36/185; A61K 31/198; A61K 31/122; A61K 36/53; A61K 36/537; A61K 31/355; A61K 31/519; A61K 31/216; A61K 31/665; A61K 9/06; A61K 9/0014; A61H 23/02; A61H 2209/00

(57) ABSTRACT

A method is provided comprising steps for (a) dispensing a topical product having a set of ingredients, wherein the set of ingredients provides pain relief to an affected area of a user; and (b) delivering the topical product via a sonic device to the affected area of a user, wherein the affected area includes joint inflammation, wherein the sonic device comprises an elongated body having an massager head receiving area designed to receive a massager head, the combination of the sonic device and the attached massager head producing a vibrating massager promoting optimal absorption of the set of ingredients designed to stimulate and increase blood flow to the joint inflammation providing a reduction of pain for the user, wherein the sonic device and topical product are packaged together in a single packaging unit.

### CROSS-REFERENCE TO RELATED APPLICATIONS

N/A

BACKGROUND OF THE INVENTION

1. Field of the Invention

The present invention relates to topical products, and more specifically an application of a topical product using a sonic device.

2. Description of Related Art

Currently, there are over 116 million chronic pain suffers in the USA alone, alongside an even greater population that suffers non-chronic pain from a variety of cases. In fact, pain affects more Americans than diabetes, heart disease, and cancer combined. For U.S. adults reporting pain, main causes include headaches, migraines, low back pain, neck pain, knee pain, shoulder pain, finger pain, and hip pain.

The U.S. pain management market is the largest single market with sales of over $13 billion. A typical solution to relieve chronic pain is by taking painkillers. However, prescription painkillers are considered a major contributor to the total number of drug related deaths. One safe and convenient alternative, is applying a topical analgesics for treating stiffness or pain in the muscles. The global market for over the counter (OTC) sales regarding topical analgesics are over $4 billion, with annual sales exceeding $800 million. Presently, there has not been any significant innovation in this field. Consequently, there is a need for a safe and effective OTC solution for pain management that is an improvement over existing methods.

### BRIEF SUMMARY OF THE INVENTION

In one embodiment of the present invention, a method is provided comprising steps (a) dispensing a topical product having a set of ingredients; and (b) delivering the topical product via a sonic device to an affected area of a user, wherein the sonic device and topical product are packaged together in a single packaging unit.

In one embodiment, in step (a), the set of ingredients comprises a pain relief composition. In one embodiment, in step (b), the sonic device comprises an elongated body having an attachment head receiving area designed to receive a plurality of attachment heads. In another embodiment, the sonic device and an attached attachment head of the plurality of attachment heads creates a vibrating massager. In yet another embodiment, the vibrating massager promotes optimal absorption of the set of ingredients.

In one embodiment, the vibrating massager stimulates and increases blood flow providing a reduction of pain for the user. In another embodiment, the attachment head receiving area comprises a cylindrical flange and cylindrical receiving space, and each of the plurality of attachment heads comprises a hollow interior having a cylindrical bottom engagement surface including a plurality of protruding ridges, wherein the plurality of protruding ridges are positioned under the cylindrical flange in the cylindrical receiving space securing an attachment head to the attachment head receiving area.

In one embodiment, the sonic device further comprises an angled indented area designed to aid in the removal of the attachment head. In another embodiment, the pain relief composition includes capsaicin. In one embodiment, the set of ingredients includes glucosamine and dimethyl sulfone. In another embodiment, the set of ingredients further includes aloe, ginkgo biloba extract, ginseng root extract, alfalfa, and lemongrass. In yet another embodiment the topical product is comprised of a gel.

In another aspect to the invention, a method is provided comprising steps (a) dispensing a topical product having a set of ingredients, wherein the set of ingredients provides pain relief to an affected area of a user; and (b) delivering the topical product via a sonic device to the affected area of a user, wherein the affected area includes joint inflammation, wherein the sonic device comprises an elongated body having an massager head receiving area designed to receive a massager head, the combination of the sonic device and the attached massager head producing a vibrating massager promoting optimal absorption of the set of ingredients designed to stimulate and increase blood flow to the joint inflammation providing a reduction of pain for the user, wherein the sonic device and topical product are packaged together in a single packaging unit.

In one embodiment, in step (a), the affect area includes muscle pain, muscle strains, and chronic pain. In another embodiment, in step (a), the set of ingredients comprises a pain relief composition having capsaicin. In another embodiment, in step (a), the set of ingredients comprises glucosamine, dimethyl sulfone, aloe, ginkgo biloba extract, ginseng root extract, alfalfa, and lemongrass. In yet another embodiment, in step (a), the set of ingredients comprises arnica montana flower extract, aesculus hippocastanum seed extract, epilobium angustifolium extract, arginine HCL, phytonadione, baloe barbadensis leaf juice. In yet another embodiment, in step (a), the set of ingredients comprises aloe barbadensis leaf juice, lavandulaangustifolia oil, salvia officinalis leaf extract, canangaodorata flower extract, anthemisnobilis flower extract, tocopheryl acetate, folic acid, retinylpalmitate, and magnesium ascorbyl phosphate. In yet another embodiment, in step (a), the topical product is comprised of a gel or cream. In one embodiment, the method further comprises a step (c) moving the sonic device in a circular motion over the affected area.

### BRIEF DESCRIPTION OF THE SEVERAL VIEWS OF THE DRAWINGS

Other features and advantages of the present invention will become apparent when the following detailed description is read in conjunction with the accompanying drawings, in which:

FIG. 1 is a top perspective view of a sonic device according to an embodiment of the present invention.

FIG. 2 is a bottom perspective view of a sonic device according to an embodiment of the present invention.

FIG. 3 is a top view of a sonic device according to an embodiment of the present invention.

FIG. 4 is a side view of a sonic device according to an embodiment of the present invention.

FIGS. 5A-C are perspective views of various attachment heads according to an embodiment of the present invention.

FIG. 6 is an application of topical product using a sonic device according to an embodiment of the present invention.

FIG. 7 is a method of using a sonic device with a topical product according to an embodiment of the present invention.

### DETAILED DESCRIPTION OF THE PREFERRED EMBODIMENT

The following description is provided to enable any person skilled in the art, to make and use the invention and sets forth the best modes contemplated by the inventor of carrying out their invention. Various modifications, however, will remain readily apparent to those skilled in the art, since the general principles of the present invention have been defined herein to specifically provide an application of topical product using a sonic device.

The present invention is particular useful for users with chronic pain and arthritis. Since joints have limited blood flow, users with arthritis show slow recovery from joint pain due to limited blood flow to their joints. Additionally, inflammation builds ups around the joint due to the decreased blood flow causing extreme pain. The present invention can help users stimulate and increase blood flow in the inflamed joint, decreasing arthritic pain. Additionally, the present invention improves quality of life, by providing, therapeutic massage with a topical product releasing serotonin, relieving pain, and healing soft tissue cells. The present invention also increases the flow of oxytocin, relaxing muscles, and providing a feeling of calmness and contentment. Further, the present invention, an application of topical product with a sonic device provides an unexpected result. Specifically, the sonic device provides vibrations which improve blood circulation and oxygen flow to painful areas while promoting optimal absorption of ingredients from the topical product. The absorption of topical product makes the present invention unique and useful for patients and users suffering from pain and injuries.

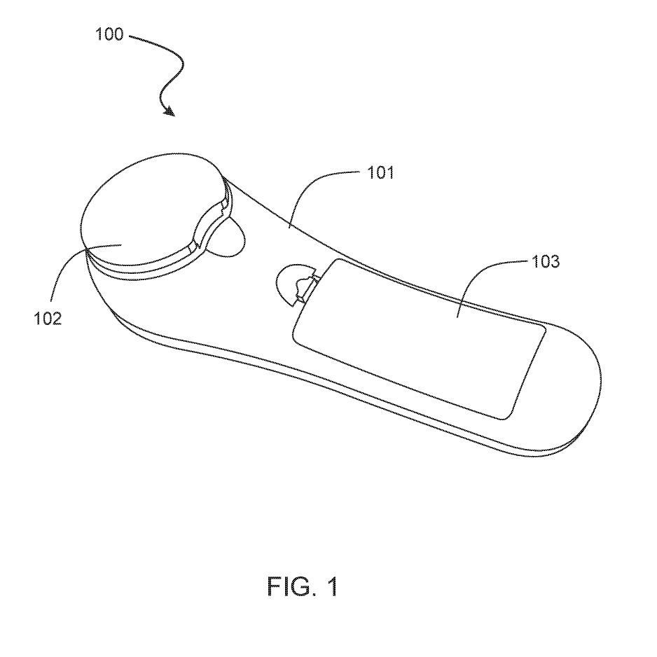

FIGS. 1-4 illustrate various views of a sonic device 100 according to an embodiment of the present invention. Referring now to FIG. 1, the sonic device comprises an elongated body 101, attachment head receiving area 102, and battery compartment 103. The elongated body has a cylindrical ergonomic design providing a comfortable and easy to grip surface. The attachment head receiving area is design to secure a plurality of attachment head designs. This will be discussed in further detail below. The battery compartment provides easy access to two required AA batteries to operate the hand-held device, as well known in the art.

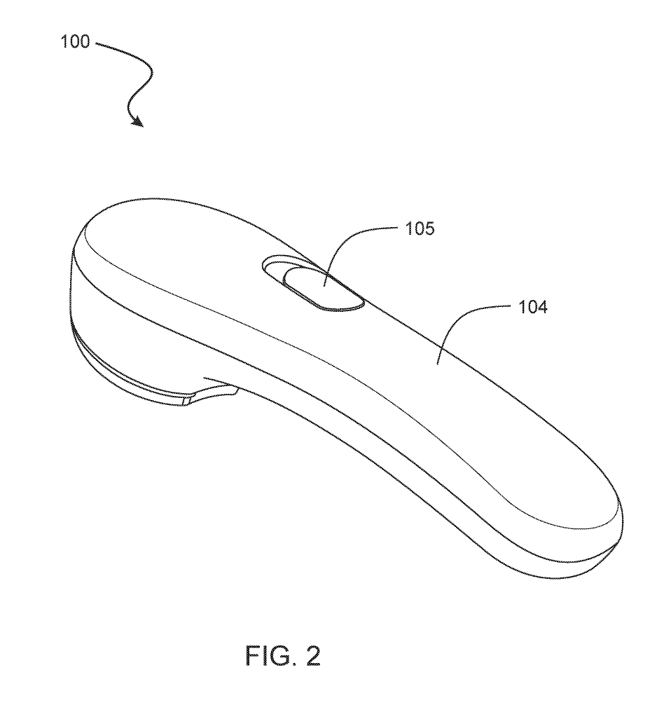

Referring now to FIG. 2, the sonic device comprises a bottom surface 104 including power controls 105. The power controls allow the sonic device to be turned on, and select a number of speed options. In some embodiments, the number of speed options is two, including a low speed and high speed. Low speed correlating to a low sonic vibration, and high speed correlating to a high sonic vibration.

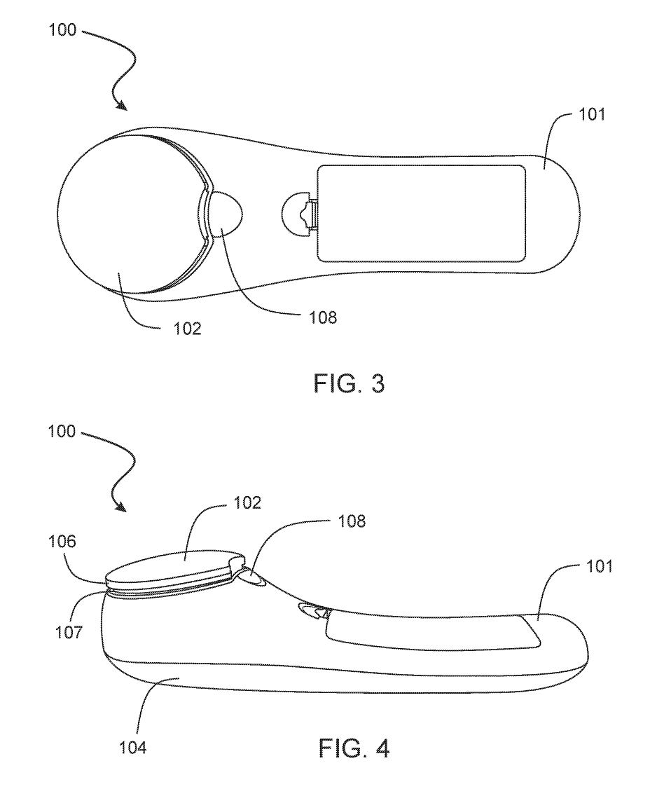

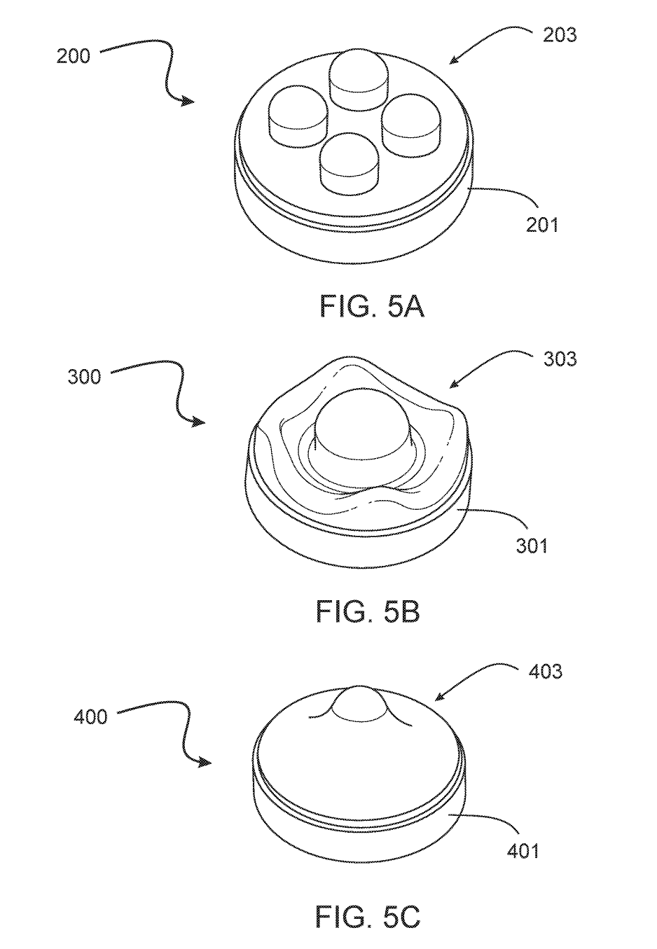

Referring now to FIGS. 3 and 4, the sonic device comprises an angled indented area 108 enabling a method of removing an installed attachment head (FIGS. 5A-C) from the device after use. The attachment head may be removed from the device for cleaning, storage, or if a different attachment head is desired. The various attachment heads will be discussed in detail below. Once a user selects a desired attachment head, which is dependent on the pain location and target areas, the desired attachment head is snapped into place on the head receiving area. Specifically, the head receiving area includes a cylindrical flange 106 and cylindrical receiving space 107. The attachment heads includes a hollow interior having a cylindrical bottom engagement surface including a plurality of protruding ridges. For installation, the engagement surface is fit over the cylindrical flange and cylindrical receiving space, and the protruding ridges are positioned under the bottom surface of the flange, securing the attachment head to the sonic device. For removal, the attachment head may be twisted off the attachment head receiving area or pried off the attachment head receiving area by use of the indented angled indented area. The pried off or pushed off method may be completed with the user's thumb. Likewise, a tool, such as a mini spatula, may be used to assist in removing the attachment head. This is particular useful if a user suffers from arthritis or other condition causing the removal process to be difficult when using their hands.

FIGS. 5A-C are perspective views of various attachment heads according to an embodiment of the present invention. Referring now to FIG. 5A, a first attachment head 200 is illustrated. The first attachment head comprises a cylindrical body 201 with a hollow interior having a cylindrical bottom engagement surface including a plurality of protruding ridges for engagement with the attachment head receiving area as previously mentioned. The cylindrical body has a plurality of raised bumps 203. The raised bumps are constructed of a cylindrical body with a spherical top. In some embodiments, the plurality of raised bumps includes four raised bumps, but it is understood that the number of raised bumps may vary. The first attachment head is intended for soothing larger muscles and sores areas including but not limited to the back, shoulders, thighs, and calf muscles of the user.

Referring now to FIG. 5B, a second attachment head 300 is illustrated. The second attachment head comprises a cylindrical body 301 with a hollow interior having a cylindrical bottom engagement surface including a plurality of protruding ridges for engagement with the attachment head receiving area as previously mentioned. The cylindrical body has a deep muscle message head 303. The deep muscle message head is constructed of a single cylindrical body with a spherical top centered on the cylindrical body surrounded by circumferential concave surfaces. The second attachment head is intended for soothing sore a shoulder, back, hand, arm, leg muscles and joints.

Referring now to FIG. 5C, a third attachment head 400 is illustrated. The third attachment head comprises a cylindrical body 401 with a hollow interior having a cylindrical bottom engagement surface including a plurality of protruding ridges for engagement with the attachment head receiving area as previously mentioned. The cylindrical body has an accupoint message head 403. The accupoint message head is constructed of a single conical point centered on the cylindrical body. The third attachment head is intended for invigorating tense muscles and smaller joints areas including fingers, wrists, shoulder joints, neck and elbows. It is understood that the attachment heads described above are merely examples of various attachment head shapes and constructions, and variations of the shapes and constructions of the attachment heads can be made without departing from the spirit and scope of the invention.

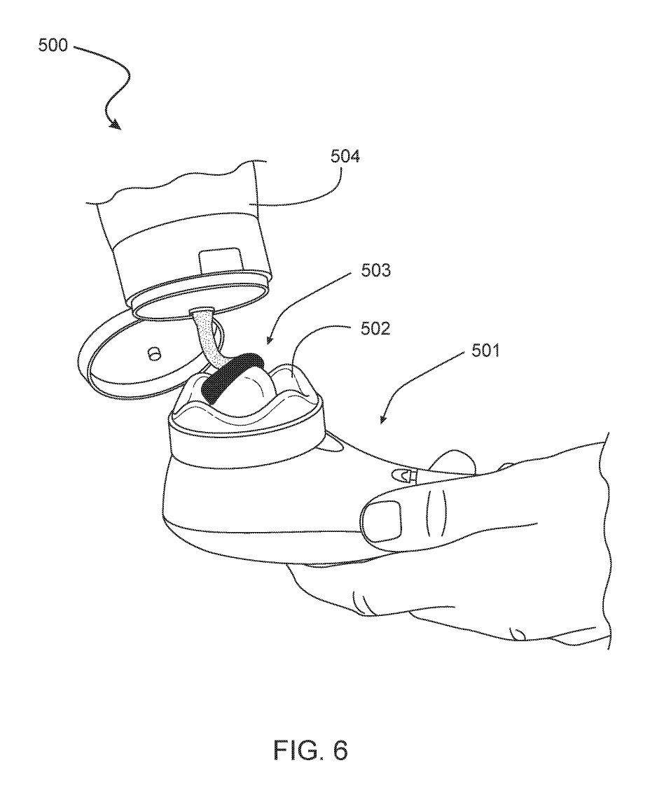

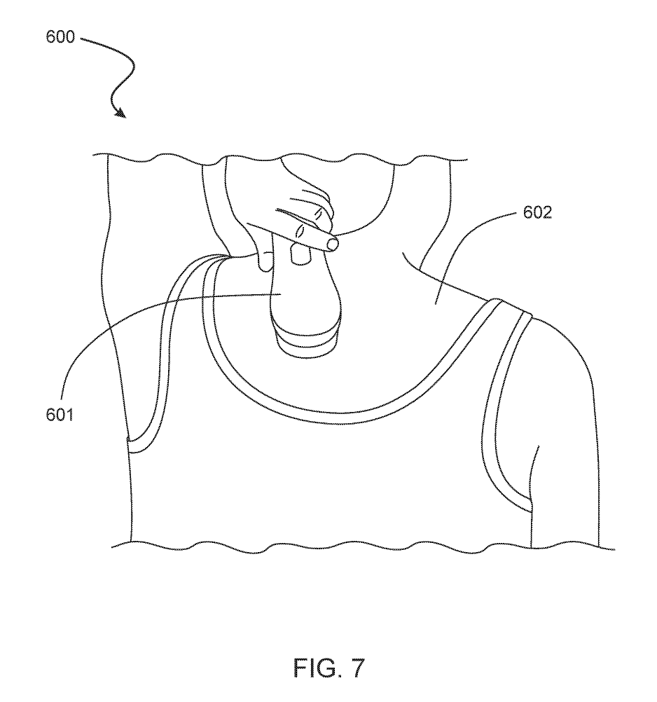

FIG. 6 is an application of topical product 503 using a sonic device 501 according to an embodiment of the present invention. In some embodiments, a topical product 503 housed in a container 504 is applied directly an attachment head 502 on the sonic device. Although a container is illustrated, the topical product may be stored in any type of containing vessel as well known in the art. In alternative embodiments, the topical product may be applied directly to the affected area of the user. Yet in another embodiment, the sonic device may be used without a topical product. The operation and use of the sonic device will be discussed in greater detail below.

The topical product may be comprised of a gel, cream, or other consistency, including but not limited to ointment, lotion, spray, foam, paste, collodion, emulsion, aerosol, solution, suspension, or other topical dosage forms and preparations. In some embodiments, the topical product comprises a pain relief gel that is greaseless, odorless, and effective for relieving a large number of pain types. The pain relief gel further comprises capsaicin providing temporary pain relief. The amount of capsaicin may vary, but preferably the pain relief gel includes 0.0625% capsicum oleoresin which contains 0.025% capsaicin. Capsaicin gives a pleasant warming sensation providing users targeted relief at the source of the pain. Research supports the use of capsaicin for osteoarthritis and rheumatoid arthritis with no drug interaction. This highly concentrated amount of capsaicin, which is FDA approved safe and effective therapy, helps to surge blood flow and circulation to the areas users need it most. Additional, the pain relief gel includes glucosamine and dimethyl sulfone (MSM), power natural ingredients that help support maximum flexibility, joint lubrication, and maintenance for healthy cartilages and connective tissues. Further, the pain relief gel may include other natural ingredients such as aloe, ginkgo biloba extract, ginseng root extract, alfalfa, and lemongrass. It is known that aloe provides hydration and moisturizes the skin, while ginkgo biloba extract has antioxidant and anti-inflammatory properties. Likewise, ginseng root extract helps the brain produce more endorphins that help to balance the physical and emotional stress hormones secreted from the adrenal glands, while also strengthening the immune system. Alfalfa extract or medicago sativa extract high in vitamins A and C, niacin, riboflavin, folic acid, and the minerals calcium, magnesium, iron, and potassium. Alfalfa also contains bioflavonoids and has anti-inflammatory properties to reduce joint stiffness and pain.

The pain relief gel may be used to treat sports injuries, arthritis, carpal tunnel syndrome, chronic and acute pain, as well as neck and head pain from tension, while improving circulation and relieving stress. The pain relief gel includes but is not limited to water, glycerin, PEG-75 lanolin, polysorbate 20, PEG-40 hydrogenated castor oil, acrylates/C10-30 alkyl acrylate crosspolymer, triethanolamine, emu oil, aloe barbadensis leaf juice, glucosamine, dimethyl sulfone, seed of celosia argentea extract, ginkgo biloba extract, panax ginseng root extract, medicago sativa (alfalfa) extract, cymbopogon schoenanthus extract, tocopheryl acetate, propylene glycol, DMDM hydantoin, methylparaben, and propylparaben.

In some embodiments, the topical product comprises an arnica pain and bruise cream designed to provide relief from bruising, bruising, strains, sprains, pain, inflammation, stiffness, and swelling. The arnica pain and bruise cream comprises arnica montana flower extract, aesculus hippocastanum (horse chestnut) seed extract, epilobium angustifolium flower/leaf/stem extract, arginine HCL, phytonadione, baloe barbadensis leaf juice. The arnica flower extract helps alleviate sprains, muscle pains and dislocations. Specifically, the arnica flower extract helps prevent the formation of bruises by stopping the capillaries from bleeding, by stimulating the activity of white blood cells which process congested blood, and by dispersing trapped fluids from joints and muscles and bumped and bruised tissue. The arnica flower extract also has anti-inflammatory and anti-bacterial qualities to help to reduce pain and swelling as well as improving wound healing. The horse chestnut extract has antioxidant, anti-inflammatory, vasoprotective, astringent, and analgesic properties to treat varicose veins, varicose eczema, frostbite, bruising, rheumatoid arthritis, sprains, muscle tension, and fluid retention. The epilobium angustifolium flower/leaf/stem extract or willow herb extract is used as a soothing, cleansing and healing agent to treat minor bumps, skin rashes, ulcers, and numerous other skin irritations. The arginine or L-arginine is an amino acid that when applied topically helps reduce inflammation and improve blood circulation. In some embodiments, the arnica pain and bruise cream comprises vitamin K, which when applied topically can limit the leaking, reducing the severity of a bruise, particularly if applied soon after the trauma. Further, when vitamin K is absorbed through the skin it can aid damaged tissue for a faster healing process. The arnica pain and bruise cream includes but is not limited to water, caprylic/capric triglyceride, cetearyl alcohol, aloe barbadensis leaf juice, isopropyl myristate, glyceryl stearate SE, arnica montana flower extract, ceteareth-20, propylene glycol, glycerin, mineral oil, dimethicone, polysorbate 20, aesculus hippocastanum (horse chestnut) seed extract, epilobium angustifolium flower/leaf/stem extract, xanthan gum, arginine HCL, phytonadione, DMDM hydantoin, methylparaben, propylparaben, and fragrance.

In some embodiments, the topical product comprises a restless leg relief cream designed to provide relief from cramps, tingling, arching, itching, pain, twitching, and restlessness in the leg regions, as well as providing relieve from pins and needles sensation in the feet. The arnica pain and bruise cream comprises aloe barbadensis leaf juice, lavandulaangustifolia (lavender) oil, salvia officinalis (sage) leaf extract, canangaodorata flower (ylangylang) extract, anthemisnobilis flower (chamomile) extract, tocopheryl acetate (vitamin E acetate), folic acid, retinylpalmitate (vitamin A palmitate), and magnesium ascorbyl phosphate (MAP). The lavender oil has antiseptic and anti-inflammatory properties designed to help heal tingling sensations and pain. Further, the primary components of lavender oil are linalool and linalyl acetate. Linalool and linalyl acetate can reducing cramps and pains associated with restless leg syndrome, as well as reducing anxiety. Sage leaf extract has many therapeutic properties, and reduces tension in smooth muscles thus reducing twitching and cramps associated with restless leg syndrome. Ylangylang extract helps reduce tension and anxiety, while calming nerves leading to a reduction of symptoms related to restless leg syndrome. Chamomile extract has anti-inflammatory properties providing a beneficial topical remedy for reducing itching and irritation. In some embodiments, the restless leg relief cream comprises water, aloe barbadensis leaf juice, polyacrylamide, C13-14 isoparaffin, laureth 7, benzocaine hcl, magnesium ascorbyl phosphate, lavandulaangustifolia (lavender) oil, salvia officinalis (sage) leaf extract, propylene glycol, canangaodorata flower (ylangylang) extract, anthmisnobilis flower (chamomile) extract, tocopheryl acetate, folic acid, retinylpalmitate, methylparaben, propylparaben, and diazolidinyl urea.

In some embodiments, the topical product comprises additional active ingredients including but not limited camphor, menthol, methyl salicylate, trolamine, and histamine dihydrochloride. It is understood that there may exist an infinite number of inactive ingredients in the various topical product compositions, and the topical product compositions are not limited to the combinations listed above. In some embodiments, the topical product is available to users over-the-counter (OTC), without a prescription. In the preferred embodiment, the topical product is packaged with the sonic device. That is, the sonic device and accompanying topical product is sold as a combination together. This provides users access to the preset invention, in one convenient purchase. In some embodiments, the sonic device is packaged with one topical product composition, with other topical product compositions available for purchase. In alternative embodiments, the sonic device is packaged with more than one topical product composition. The packaging may be any packing type, known in the art, including but not limited to a cardboard box, reusable bag, plastic based packaging, paper-based packaging, metal based packaging, or travel container.

FIG. 7 is a method 600 of using a sonic device 601 with a topical product according to an embodiment of the present invention. Referring now to FIG. 7, the sonic device with topical product is positioned on an affected area of a user 602. The topical product is either applied directly to the device (FIG. 6) or applied directly to the affected area. The user selects a desired speed using the powered controls 105 (FIG. 2), then gently moves the device over the affected area using a small circular motion. Depending on the affected area location, and the specific result desired, any of the attachment heads may be used. For exemplary purposes, the device on a user's back is illustrated, but it is understood that the affected area may be at any location on the user, including the locations previously mentioned. For optimal results, the sonic device with topical product should be used multiple times a day, every day, preferably 3-4 sessions a day for 4-6 weeks. The combination of the sonic device and topical product provides a technology to improve ingredient penetration and effectiveness into underlying skin layers. This will also accelerate and improve blood flow to the affected and application areas.

Although the invention has been described in considerable detail in language specific to structural features, it is to be understood that the invention defined in the appended claims is not necessarily limited to the specific features or acts described. Rather, the specific features and acts are disclosed as exemplary preferred forms of implementing the claimed invention. Stated otherwise, it is to be understood that the phraseology and terminology employed herein, as well as the abstract, are for the purpose of description and should not be regarded as limiting. Therefore, while exemplary illustrative embodiments of the invention have been described, numerous variations and alternative embodiments will occur to those skilled in the art. Such variations and alternate embodiments are contemplated, and can be made without departing from the spirit and scope of the invention

It should further be noted that throughout the entire disclosure, the labels such as left, right, front, back, top, bottom, forward, reverse, clockwise, counter clockwise, up, down, or other similar terms such as upper, lower, aft, fore, vertical, horizontal, oblique, proximal, distal, parallel, perpendicular, transverse, longitudinal, etc. have been used for convenience purposes only and are not intended to imply any particular fixed direction or orientation. Instead, they are used to reflect relative locations and/or directions/orientations between various portions of an object.

In addition, reference to “first,” “second,” “third,” and etc. members throughout the disclosure (and in particular, claims) are not used to show a serial or numerical limitation but instead are used to distinguish or identify the various members of the group.

What is claimed is:

1. A method comprising steps:
(a) dispensing a topical product having a set of ingredients; and
(b) delivering the topical product via a sonic device to an affected area of a user, wherein the sonic device and topical product are packaged together in a single packaging unit.

2. The method of claim 1, wherein in step (a), the set of ingredients comprises a pain relief composition.

3. The method of claim 1, wherein in step (b), the sonic device comprises an elongated body having an attachment head receiving area designed to receive a plurality of attachment heads.

4. The method of claim 3, wherein the sonic device and an attached attachment head of the plurality of attachment heads creates a vibrating massager.

5. The method of claim 4, wherein the vibrating massager promotes optimal absorption of the set of ingredients.

6. The method of claim 4, wherein the vibrating massager stimulates and increase blood flow providing a reduction of pain for the user.

7. The method of claim 3, wherein the attachment head receiving area comprises a cylindrical flange and cylindrical receiving space, and each of the plurality of attachment heads comprises a hollow interior having a cylindrical bottom engagement surface including a plurality of protruding ridges, wherein the plurality of protruding ridges are positioned under the cylindrical flange in the cylindrical receiving space securing an attachment head to the attachment head receiving area.

8. The method of claim 7, wherein the sonic device further comprises an angled indented area designed to aid in the removal of the attachment head.

9. The method of claim 2, wherein the pain relief composition includes capsaicin.

10. The method of claim 9, wherein the set of ingredients includes glucosamine and dimethyl sulfone.

11. The method of claim 10, wherein the set of ingredients further includes aloe, ginkgo biloba extract, ginseng root extract, alfalfa, and lemongrass.

12. The method of claim 11, wherein the topical product is comprised of a gel.

13. A method comprising steps:
(a) dispensing a topical product having a set of ingredients, wherein the set of ingredients provides pain relief to an affected area of a user; and
(b) delivering the topical product via a sonic device to the affected area of the user, wherein the affected area includes joint inflammation, wherein the sonic device comprises an elongated body having an massager head receiving area designed to receive a massager head, the combination of the sonic device and the attached massager head producing a vibrating massager promoting optimal absorption of the set of ingredients designed to stimulate and increase blood flow to the joint inflammation providing a reduction of pain for the user, wherein the sonic device and topical product are packaged together in a single packaging unit.

14. The method of claim 13, wherein in step (a), the affect area includes muscle pain, muscle strains, and chronic pain.

15. The method of claim 13, wherein in step (a), the set of ingredients comprises a pain relief composition having capsaicin.

16. The method of claim 13, wherein in step (a), the set of ingredients comprises glucosamine, dimethyl sulfone, aloe, ginkgo biloba extract, ginseng root extract, alfalfa, and lemongrass.

17. The method of claim 13, wherein in step (a), the set of ingredients comprises arnica montana flower extract, aesculus hippocastanum seed extract, epilobium angustifolium extract, arginine HCL, phytonadione, baloe barbadensis leaf juice.

18. The method of claim 13, wherein in step (a), the set of ingredients comprises aloe barbadensis leaf juice, lavandulaangustifolia oil, salvia officinalis leaf extract, canangaodorata flower extract, anthemisnobilis flower extract, tocopheryl acetate, folic acid, retinylpalmitate, and magnesium ascorbyl phosphate.

19. The method of claim 13, wherein in step (a), the topical product is comprised of a gel or cream.

20. The method of claim 13, further comprising a step (c) moving the sonic device in a circular motion over the affected area.
